(19) United States
(12) Patent Application Publication
(10) Pub. No.: US 2025/0281337 A1
(43) Pub. Date: Sep. 11, 2025

(54) DIAPER WITH COLLECTION POUCH

(71) Applicant: EASYDAY HEALTH PRODUCTS INC., Blainville (CA)

(72) Inventors: Cristian STEFU, Blainville (CA); Ioana Mihaela HIDISAN, Blainville (CA); Regent RACINE, Blainville (CA)

(21) Appl. No.: 18/862,733

(22) PCT Filed: May 2, 2023
(86) PCT No.: PCT/CA2023/050592

§ 371 Date: Nov. 4, 2024

Related U.S. Application Data
(60) Provisional application No. 63/337,491, filed on May 2, 2022.

(51) Int. Cl. A61F 13/495; A61F 13/49; A61F 13/58

(52) U.S. Cl. CPC A61F 13/495; A61F 13/49001; A61F 13/49019; A61F 13/58; A61F 2013/4953; A61F 2013/586

(57) ABSTRACT

A diaper comprises an outer side and an opposite inner side comprising an absorbent part. A top layer is sealed to the inner surface about its peripheral border and defines a top layer area delimited by the peripheral border that overlies at least a portion of the absorbent part but is not unconnected thereto. The top layer area, the absorbent part and the peripheral border define a pouch therebetween. The pouch defines a pouch cavity therein. The top layer area defines a body contacting surface and provides an opening for accessing the pouch cavity. When the diaper is worn by a user. the opening is provided to be positioned about the anus of the user for providing for faecal matter to enter the pouch cavity during defecation.

### CROSS-REFERENCE TO RELATED APPLICATIONS

The present application claims priority on U.S. Provisional Patent Application Ser. No. 63/337,491 filed on May 2, 2022 and incorporated herein by reference in its entirety.

### TECHNICAL FIELD

The present disclosure generally relates to diapers. More particularly but not exclusively the present disclosure relates to a diaper with a collection pouch.

### BACKGROUND

Diapers include both disposable baby diapers and adult diapers. Diapers can be necessary for adults with various conditions, such as incontinence, mobility impairment, severe diarrhea or dementia. A diaper is a type of underwear that allows the wearer to defecate or urinate without the use of a toilet, by absorbing or containing waste products to prevent soiling of outer clothing or the external environment. When diapers become soiled, they require changing, generally by a second person such as a parent or a caregiver. Diapers have a facing layer to be brought into contact with the wearer's skin, an absorbent panel adjacent thereto, and a water-impervious or a water repellant outer layer. Following defecation, prolonged contact of feces with the wearer's skin may have adverse health consequences such as rashes or infections. For caregivers, changing the soiled diapers of adult patients poses many practical challenges as the task is usually extremely messy with faecal matter spilling out of the diapers and onto the sheets of the patient's bed. The ordeal worsens with patients suffering with sever diarrhea and other digestive issues.

The present application is concerned with improvements to the diaper collection pouch as described in International Patent Application No. PCT/CA2020/000028 filed on Mar. 16, 2020, and incorporated herein by reference in its entirety. The foregoing document teaches a diaper that comprises a bottom layer and a top layer mounted thereto. The bottom layer defines a top surface and an opposite outer surface and comprises an absorbent core therebetween. The top layer is mounted to the bottom layer about a peripheral border thereof. The top layer defines a top layer area delimited by the peripheral border overlying the top surface and being unconnected to the bottom layer. The top layer area, the top surface and the peripheral border define a pouch therebetween. The top layer area defines a body contacting surface and an opening for providing access to the pouch. When the diaper is worn by the user, the opening is positioned about the anus of a user for providing for faecal matter to enter the pouch during defecation.

### OBJECTS

An object of the present disclosure is to provide a diaper with a collection pouch.

### SUMMARY

In accordance with an aspect of the present disclosure, there is provided a diaper comprising: an outer sider and an opposite inner side comprising an absorbent part; and a top layer defining a peripheral border thereof and being sealed to the inner side about the peripheral border thereof and defining a top layer area delimited by the peripheral border overlying at least a portion of the absorbent part and being unconnected thereto, the top layer area, the absorbent part and the peripheral border defining a pouch therebetween, the pouch defining a pouch cavity therein, the top layer area defining a body contacting surface and providing an opening for access to the pouch cavity; wherein when the diaper is worn by a user, the opening is provided to be positioned about the anus of the user for providing for faecal matter to enter the pouch cavity during defecation

In an embodiment, the opening is provided along a central line area of the top layer area.

In an embodiment, the top layer area comprises adhesive at each lateral side of the opening, the adhesive providing for adhering the user's skin at an inner side of each buttock within the intergluteal cleft thereby bringing the opening in close proximity to the anus to receive faecal matter directly therein. In an embodiment, the adhesive is in the form of a pair of longitudinal strips along a longitudinal length of the top layer area so as to be removably sealed along a longitudinal length of the user's skin. In an embodiment, the adhesive provides a seal with the user's skin about each lateral side of the anus and the opening substantially blocking faecal matter from flowing onto an outer surface of the top layer area beyond the seal thereby allowing faecal matter to flow into the opening. In an embodiment, the adhesive for adhering the user's skin at an inner side of each buttock within the intergluteal cleft is covered by a removable liner prior to use.

In an embodiment, the diaper comprises adhesive on the top layer for providing the top layer for adhering to the user's skin directly on each buttock outside the intergluteal cleft so as to maintain the diaper in position during movement of the user. In an embodiment, the adhesive for adhering to the user's skin directly on each buttock outside the intergluteal cleft is provided in the form of elongate strips on the top layer for providing substantial areas of the top layer without any adhesive. In an embodiment, the adhesive for adhering to the user's skin directly on each buttock outside the intergluteal cleft is covered by a removable liner prior to use.

In an embodiment, the top layer area comprises a tear line for being selectively torn along a length thereof to selectively form the opening. In an embodiment, the tear line is formed along a central line area of the top layer area for being selectively torn along a desired position of the central line area and along a desired position of the central line area. In an embodiment, the tear line comprises weak lines or perforations, the tear line defining a central portion interposed by front and rear portions, wherein the central portion has a greater number of the weak lines or the perforations than either one of the front and rear portions.

In an embodiment, the top layer provides for allowing a front part of the absorbent part to be uncovered and thus in direct contact with the user for receiving urine discharge to be absorbed, thereby substantially avoiding urine discharge from into the pouch and mixing with the feces collected therein. In an embodiment, the pouch defines a front part and a rear part, the front part defining a pair of front separate fingers defining an inwardly recessed portion therebetween exposing the front portion of the absorbent part in direct contact with the user for receiving urine discharge to be absorbed.

In an embodiment, the top layer comprises adhesive for adhering to the users skin and for adhesively sealing the inner side when folding the diaper after use thereby sealing the pouch containing the collected faecal matter.

In an embodiment, the inner side comprises a longitudinal tension along the length thereof that is greater than the a lateral tension along the width thereof.

In an embodiment, the top layer area defines a length thereof and forms waves defining longitudinal peaks and valleys along the length thereof, wherein the opening is provided at a selected one of the longitudinal peaks.

In an embodiment, the peripheral boundary of the top layer defines lateral sides thereof, wherein at least a portion of the lateral sides of the peripheral boundary are cut in a zigzag configuration.

In an embodiment, the top layer comprises soft elastic elements running along a length thereof.

In an embodiment, the top layer area comprises silicone circumscribing the opening.

Other objects, advantages and features of the present disclosure will become more apparent upon reading of the following non-restrictive description of illustrative embodiments thereof, given by way of example only with reference to the accompanying drawings.

BRIEF DESCRIPTION OF THE DRAWINGS

In the appended drawings:

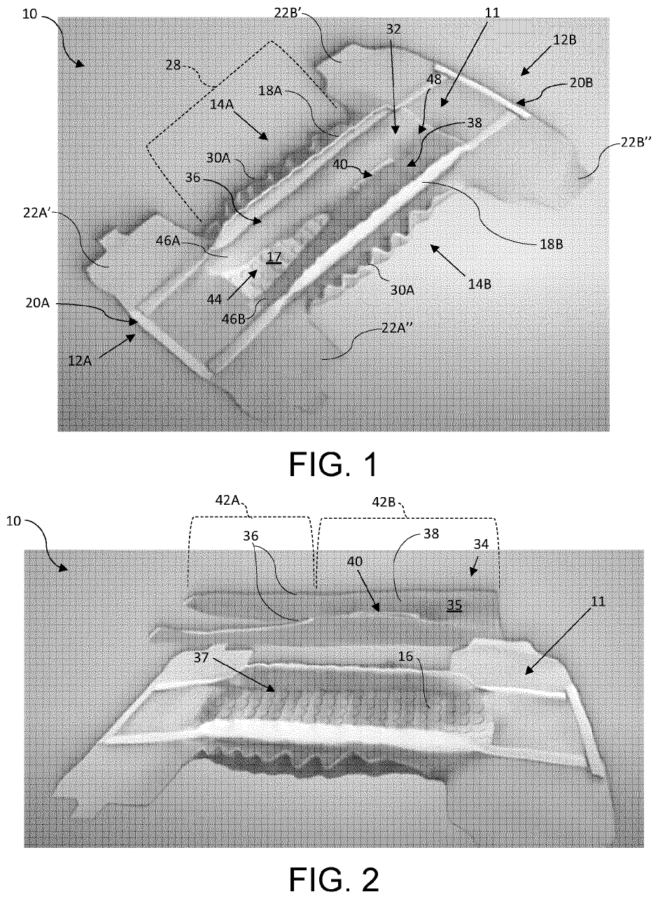

FIG. 1 is a top, perspective view of the diaper in an open position showing the inner side thereof in accordance with a non-restrictive illustrative embodiment of the present disclosure;

FIG. 2 is a top and side, perspective view of the diaper as shown in FIG. 1 in a partially exploded position thereof showing the top layer sheet being separate from the inner side thereof in accordance with a non-restrictive illustrative embodiment of the present disclosure;

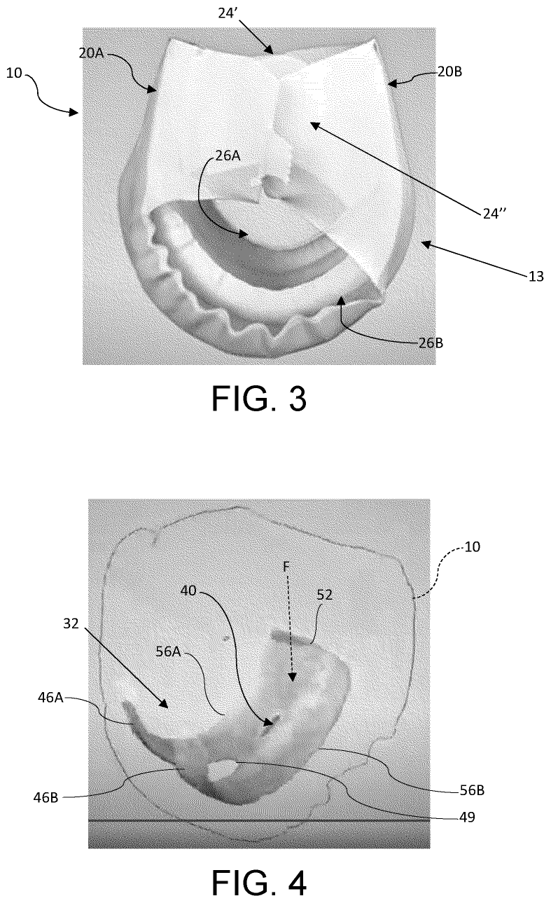

FIG. 3 is a side perspective view of the diaper of FIG. 1 in a closed position in accordance with a non-restrictive illustrative embodiment of the present disclosure;

FIG. 4 is a transparent view of the diaper as shown in FIG. 3 following use to show a perspective view of the pouch when filled in accordance with a non-restrictive illustrative embodiment of the present disclosure;

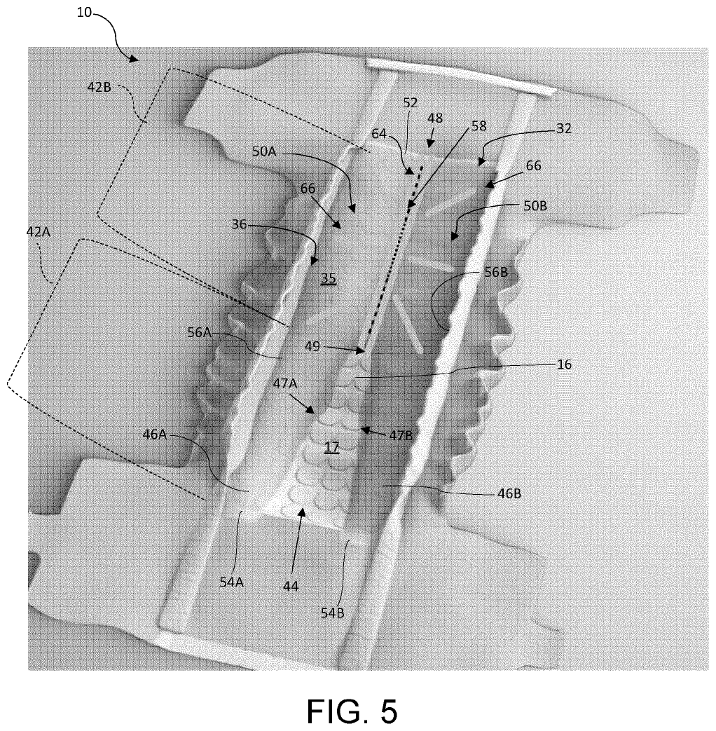

FIG. 5 is a top, perspective view of the diaper in an open position showing the inner side thereof including tear lines and adhesive in accordance with a non-restrictive illustrative embodiment of the present disclosure;

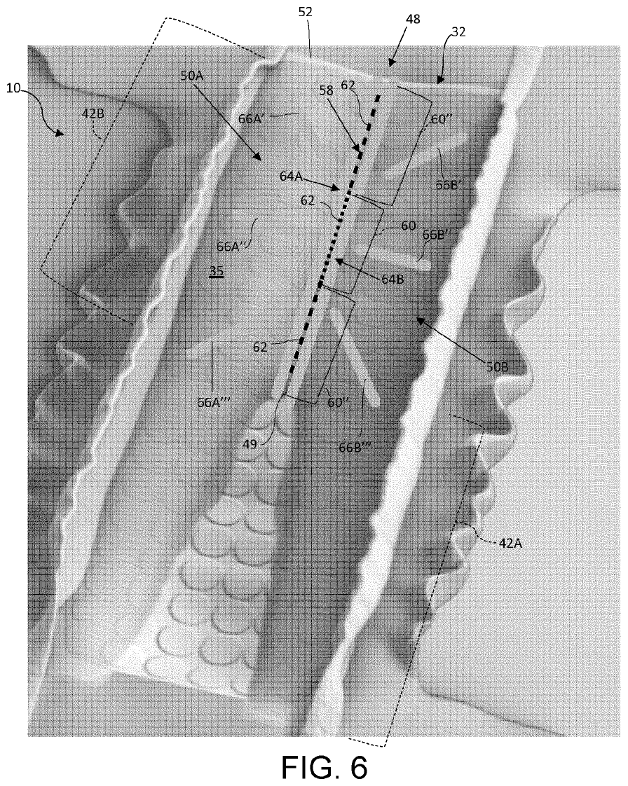

FIG. 6 is an enlarged view of FIG. 5;

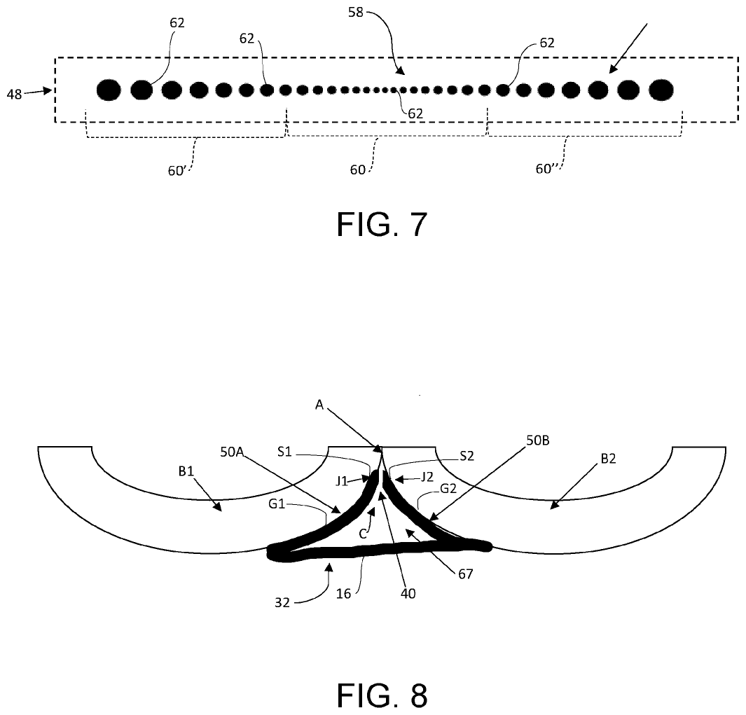

FIG. 7 is a schematic view of the central line area of the pouch of FIGS. 5 and 6 including the tear line in accordance with a non-restrictive illustrative embodiment of the present disclosure;

FIG. 8 is a schematic view of the pouch of the diaper positioned about the buttocks of a user in accordance with a non-restrictive illustrative embodiment of the present disclosure;

FIG. 9 is a top, perspective view of the diaper of FIG. 5 including removable liner strips on the adhesive on the inner side thereof in accordance with a non-restrictive illustrative embodiment of the present disclosure;

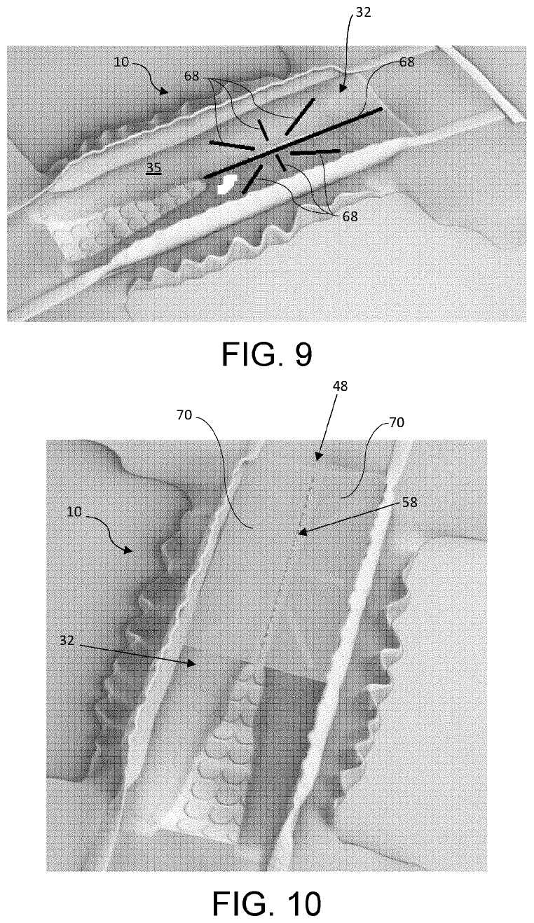

FIG. 10 is a top, perspective view of the diaper of FIG. 5 including removable liner sheets on the adhesive on the inner side thereof in accordance with a non-restrictive illustrative embodiment of the present disclosure

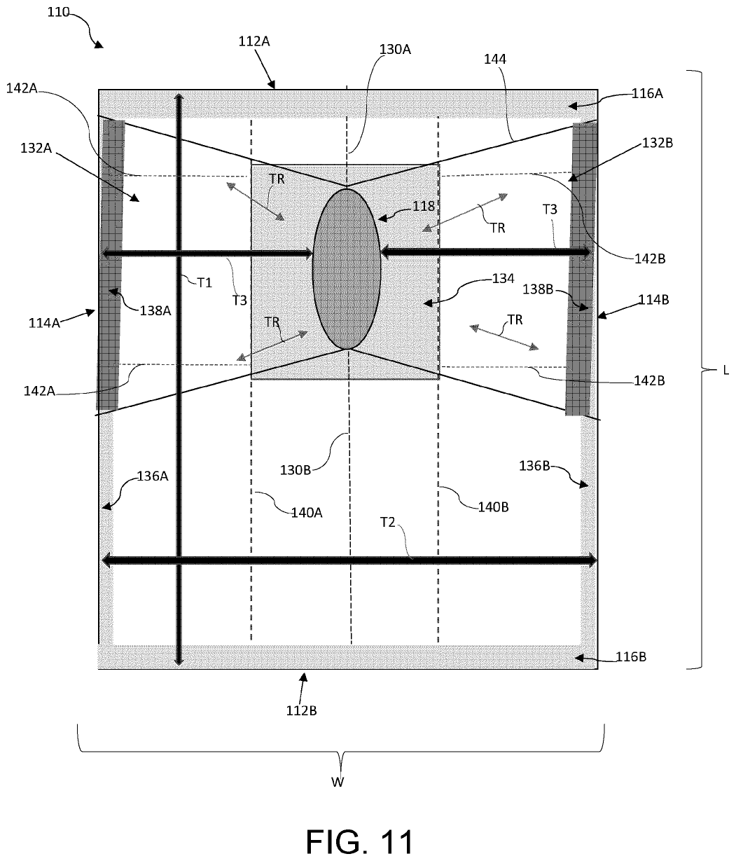

FIG. 11 is a schematic top view representation of the diaper in accordance with a non-restrictive illustrative embodiment of the present disclosure;

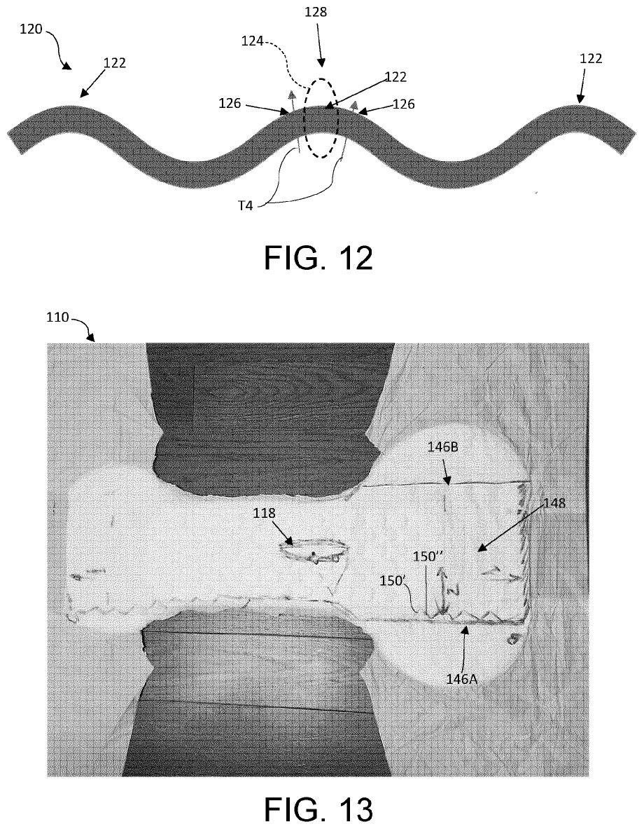

FIG. 12 is a schematic cross-sectional representation of the top layer of the diaper of FIG. 11 under stretching tension and showing the portion being cut for creating the opening leading to the pouch of the diaper;

FIG. 13 is a top plan view of the diaper in in accordance with a non-restrictive illustrative embodiment of the present disclosure;

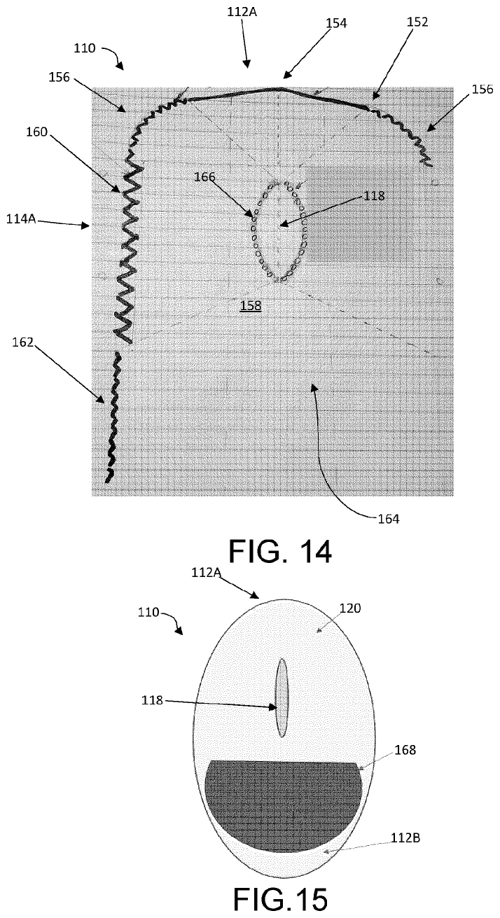

FIG. 14 is a schematic top view representation of the diaper in accordance with a non-restrictive illustrative embodiment of the present disclosure; and

FIG. 15 is a schematic top view representation of the diaper in accordance with a non-restrictive illustrative embodiment of the present disclosure.

### DETAILED DESCRIPTION OF ILLUSTRATIVE EMBODIMENTS

Generally stated and in accordance with an embodiment, there is provided a diaper comprising an outer side and an opposite inner side comprising an absorbent part. A top layer is sealed to the inner surface about its peripheral border and defines a top layer area delimited by the peripheral border that overlies at least a portion of the absorbent part but is not unconnected thereto. The top layer area, the absorbent part and the peripheral border define a pouch therebetween. The pouch defines a pouch cavity therein. The top layer area defines a body contacting surface and provides an opening for accessing the pouch cavity. When the diaper is worn by a user, the opening is provided to be positioned about the anus of the user for providing for faecal matter to enter the pouch cavity during defecation.

With reference to the Figures, non-restrictive illustrative embodiment of the present disclosure will now be described.

With reference to FIGS. 1, 2 and 3, the diaper 10 includes an inner side 11 and an outer side 13 thereof. FIG. 1 show the diaper in the open position and FIG. 3 shows the diaper in the closed position when fitted on the user.

The diaper 10 has front and rear ends, 12A and 12B, respectively, and opposite lateral sides 14A and 14B. The inner side 11 includes a bottom layer comprising an absorbent part 16 providing for receiving human discharge including menses, urine and faeces. The absorbent part 16 is flanked by leak guards 18A and 18B at each side 14A and 14B.

As is known in the art, the absorbent part 16 is layered and includes a top sheet that comes into contact with the skin of the user, an acquisition distribution layer and an absorbed core all of which are supported by the diaper chassis including the back sheet defining the outer side 13.

The front and rear ends 12A and 12B define the front and rear parts 20A and 20B of the diaper 10. Each part 20A, 20B has respective lateral side wings 22A′, 22A″ and 22B′, 22B″ which include adhesive or other fastening elements. In the closed position, wings 22A′ and 22B′ are fastened together to define one lateral part 24′ (at side 14A) of the diaper and wings 22A″ and 22B″ are fastened together to define the other lateral part 24″ (at side 14B). In the closed position, the diaper 10 defines leg opening 26A and 26B at sides 14A and 14B, respectively.

With particular reference to FIG. 1, the median section 28 of the diaper 10 interposed between the front and rear parts 20A and 20B comprises leg cuffs 30A and 30B at each side 14A and 14B, respectively which form the openings 26A and 26B together with lateral parts 24′ and 24″.

Diaper 10 includes a pouch or pocket 32 formed of a top layer or sheet 34 overlying the absorbent part 16. The layer 34 defines a peripheral edge section 36 thereof that is adhesively mounted to the inner side 11.

More particularly, the peripheral edge section 36 is adhesively mounted about and/or on or near the peripheral edge 37 of the absorbent part 16 to be sealed thereto allowing the main portion 38 of the sheet 34 to be loosely resting on the absorbent part thereby forming a pouch or pocket cavity therewith. As such, the edge section 36 can be mounted on an area of the inner side 11 surrounding the absorbent part 16 or can be mounted directly on or near the edge 37 of the absorbent part 17 or both as can be contemplated by the skilled artisan. The purpose of the top layer or sheet 34 is to be adhesively mounted to the inner side 11 of the diaper 10 about its peripheral border edge 36 and to overly the absorbent part 16 and to define a top layer area or main portion 38 which overlies at least a portion of the the absorbent part but is unconnected thereto so as to form a pouch 32 with the inner side 11 including at least a portion of the absorbent part 16.

An opening 40 is formed through the sheet 34 about the area where the anus of the user located when worn and leading to the pouch cavity for allowing faecal matter F to be collected within the pouch 32 via opening 40 (as represented in FIG. 4) thereby filling the pouch 32 during user defecation.

The sheet 34 has a front part 42A and a rear part 42B which define the front and rear parts 42A, 42B of the pouch 32 (as shown in FIG. 5) when the sheet 34 is mounted to the diaper 10 as provided herein. The front part 42A comprises a front recessed portion 44 in the form of a V-like or U-like cut-out defining a pair of laterally elongated fingers 46A and 46B at each side 14A and 14B respectively exposing a portion 17 of the absorbent part 16 therebetween which is not covered by the sheet 34.

As better shown in FIG. 5, the fingers 46A and 46B have inner edges 47A and 47B which form part of peripheral edge section 36 and which meet at a common juncture 49 therebetween (see also FIG. 4). The inner edges 47A, 47B and the common juncture 49 form a valley configuration which comprises the portion 17. The common juncture 49 defines the floor of the valley.

Portion 17 of absorbent part 16 is so positioned to directly receive urine discharged by the user and as such is direct contact with the user's body corresponding to the genitalia area of the user i.e. the penis/vagina of the user depending on the gender thereof. The foregoing avoids for urine discharge onto open absorption portion 17 to mix with faecal discharge F collected in the pouch 32. Indeed, portion 17 may include additional layers of absorbent material as can be contemplated by the skilled artisan within the scope of the present disclosure.

As shown in FIGS. 5 and 6, the rear part 42B has a generally rectangular configuration and defines a central line 48 flanked by two halves 50A and 50B. The opening 40 is formed about the central line area 48 (see also FIG. 1). The rear edge 52 (see also FIG. 4) of the rear part 42B forms part of the peripheral edge section 36. The fingers 46A and 46B define respective front ends 54A and 54B and form part of the peripheral edge section 36. The pouch 36 includes lateral edges 56A and 56B (see also Figure 4) at each lateral side 14A and 14B respectively which form part of the peripheral edge section 36.

With reference to FIGS. 5 and 6, diaper 10, in accordance with an embodiment thereof, includes a tear line 58 along a portion or the full central line area 48. The central line area 48 runs from juncture 49 to the rear edge 52 and the tear line 58 can run along the full length of the line area 48 or a portion of it. At least one portion of tear line 58 is situated at a portion 60 (see FIGS. 6 and 7) of the line area 48 that corresponds to the anus of the user, usually the middle part of the line area 48. As mentioned, the tear line 58 can extend beyond portion 60 defining front and rear portions 60′ and 60″ respectively.

The tear line 58 defines perforations 62 which allow a user to open a portion of the tear line 58 in order to form an opening 40 having the desired length and the desired position. The user can for example, pull about the tear line 58 on a portion of the sheet 34 at halves 50A and 50B or otherwise tear a desired portion of the line 58 in order to form an opening 40 at a desired position along the line area 49. Since humans have different anatomies, the position of the anus and the configuration of the intergluteal cleft differs from person to person and thus the position and size of the opening 40 may need to vary from one individual to another. Having tear lines allows the caregiver to selectively make an appropriate opening 40 with a desired length and position along the line area 49 depending on the anatomical particularity of the individual wearing the diaper 10.

In an embodiment and with reference to both FIGS. 6 and 7, there are relatively more and larger perforations or longer weak lines 62 at middle portion 60 and relatively lesser and smaller perforations or shorter weak lines 62 at either one of the front and rear portions 60′ and 60″. Indeed, since in most cases, the opening 40 will most likely fall within section 40, the greater number of perforations/weak lines 62 provide for tearing the tear line 58 at smaller increments to allow a greater number of possible opening 40 positions than would be required in portions 60′ and 60″.

In some cases, the weak lines or perforations 62 may not be sufficiently visible and the sheet 34 may be marked with indications similar to the schematic illustration of FIG. 7 to provide just one example in order to more clearly show where the weak lines or perforations are positioned as well as their numerical concentration.

Indeed, the tear line 58 may be configured in a variety of ways known in the art within the context of the present disclosure providing for conveniently tearing the sheet 34 to make the opening 40.

Sheet 34 defines the outer or top surface 35 of the pouch 32 which will be in contact with the wearer's skin.

As shown in FIGS. 5 and 6, the pouch 32 comprises an adhesive 64 on its outer surface 35 at each lateral side of the tear line 58. The adhesive 64 is provided in the form of two adhesive strips 64A and 64B flanking the tear line 58 and running longitudinally along the length of the central line area 48. In an embodiment, the adhesive 64 is a low-density adhesive configured for adhering to human skin (such as edible glue for example). Each strip 64A, 64B adheres to a respective buttock of the wearer along the inner side thereof within the intergluteal cleft.

As represented in FIG. 8, the adhesive strips 64A, 64B are sealed to the inner sides S1, S2 of the buttocks B1, B2 respectively within the intergluteal cleft C bringing the opening 40 towards the anus A. In this way, during defecation, feces are blocked from the spilling unto the outer surface 35 by the sealed junction J1 between adhesive strip 64A and the inner buttock side S1 and the sealed junction J2 between adhesive strip 64B and the inner buttock side S2.

Turning now to FIGS. 5 and 6 and accordance with an embodiment of the diaper 10, the outer surface 35 of the pouch 32 also comprises additional adhesive material 66 on the sheet halves 50A and 50B for adhering to the outer surface (glute area) the buttocks. In the embodiment, the adhesive material 66 comprises strips of adhesive radially extending relative to the central portion line area 48. In the example shown here, sheet half 50A comprises an rear upwardly diagonally extending adhesive strip 66A′, a middle laterally extending adhesive strip 66A″, a front downward diagonally extending adhesive strip 66A′″. The sheet half 50B comprises strips that are essentially a mirror image of those of sheet half 50B, namely a rear upwardly diagonally extending adhesive strip 66B′, a middle laterally extending adhesive strip 66B″, a front downward diagonally extending adhesive strip 66B′″. Of course, the skilled artisan will readily appreciate that adhesive 66 can be positioned on surface 35 in a variety of suitable ways within the context of the present disclosure.

Turning back to FIG. 7, the adhesive 66 provides for adhesively sealing each half 50A, 50B to a respective outer surface (glute area) G1 and G2 of each buttock B1, B2, respectively. Thus, the foregoing allows to maintain the sheet 34 in position with the opening 40 near the anus blocking spillage of feces between surface 34 and the buttocks of the user and allowing the feces to flow into he pouch cavity 67 (see FIG. 8) via opening 40. Maintaining the sheet 34 in place is especially required during movement of the user.

In an embodiment, the opening 40 (and the remaining untorn tear line 58 if the whole tear line 58 has not been torn) provides for less tension in the central line area 48 (relative to the tension in the sealed edge portion 36) moving it upwards into the intergluteal cleft C further helping position the opening 40 at anus A and providing a synergistic effect to the sealing action of the adhesives 64 and 66 as described hereinabove.

In an embodiment and as shown in FIG. 9, the adhesives 64 and 66 are covered by removal liner films 68 which are removed prior to diaper use.

In an embodiment and as shown in FIG. 10, the adhesives 64 and 66 are covered by removal sheets 70 which are removed prior to diaper use. In an embodiment each of pair of sheets 70 is positioned on a respective half 50A, 50B of the surface 35 without covering the tear line 58 therebetween.z

When the caregiver removes the diaper 10 from the user following defecation and filling of the pouch 32 with faecal matter F as shown in Figure, the diaper provides for avoiding any faecal matter from spilling unto surface 35 or anywhere in the inner side 11 thereby not exposing the caregiver to the faecal matter and moreover, the caregiver can fold the diaper with it's the inner sides of its front and rear parts 42A and 42B meeting or about the central line area 48 as is convenient to enclose the inner side 11 and close the opening 40. When folding the adhesives 64 and 66 will provide for adhering to parts of the inner side 11 they interface with thereby sealing the diaper 10 so as to fully enclose the inner side 11 and seal the pouch 32 with only the outer side 13 being exposed. The foregoing provides for exposing the caregivers to the toxic gases of the faecal matter which are a health hazard.

With reference to FIG. 11, there is shown a top schematic view of a diaper 110 have front and rear ends, 112A and 12B, respectively, and opposite lateral sides 114A and 114B. As taught in PCT/CA2020/000028, the diaper 110 includes top and bottom layers that are glued together at top and bottom edge portions 116A and 116B respectively. As taught in PCT/CA2020/000028 the diaper 110 includes an opening 118 leading to a collection pouch.

In an embodiment, the strength of the glued portions 116A and 116B provide a longitudinal tension T1 that extends throughout the length L of the diaper. This tension T1 is related to the length of the diaper 110 vs the width W of the diaper 110. In an embodiment, the vertical tension T1 is two to three times greater that the lateral tension T2 across the width of the diaper. Having a longitudinal tension T1 that is greater than the lateral tension is convenient for avoiding the diaper 110 to fold inwardly bringing in sides 114A and 114B closed together. Furthermore, the greater vertical tension T1 provides the C shaped curve to the diaper 110. As ends 112A and 112B are pulled inwardly, the shorter length of the inner layer creates a gap with the longer bottom layer thus forming the collection pouch.

During manufacturing, the bottom later will be stretched out more vertically than laterally and then the inner layer will be glued onto the bottom layer providing the desired strength at the glued portions 116A and 116B which further provides the tension T1.

Turning to FIG. 12, when the top layer (or liner) 120 is stretched to be glued onto the bottom layer as explained above, tension waves 122 are created along the longitudinal length of the top layer. The tension waves are formed by selected stretching tension and their position can be selectively adjusted by modulating the stretching tension. It is convenient to cut a portion 124 of the material 120 to form the opening 118 at a tension wave 122 as the material 126 surrounding this portion 124 (and thus the opening 118) is upwardly rising forming an apex 128 at the opening 118. This apex finds its way into the intergluteal cleft of the user and thus provides for positioning the opening 118 at the anus.

Turning back to FIG. 11, the diaper 110 includes, in an embodiment thereof, central soft elastic elements 130A and 130B respectively sewed into the top layer 120 from end 112A to the opening 118 and from the opening 118 to the end 112B. These elastic elements 130A and 130B form an apex that upwardly extends along their length which like the apex 128 finds its way into the cleft. In an embodiment, the elastics 130A and 130B reinforce apex 128.

Indeed, the foregoing apex-forming configuration allows for guiding the inner layer 120 into the cleft and thereby positioning the opening at the anus of the wearer. In fact, the inner layer 120 is not fully connected to the bottom layer and thus there is discrepancy in the lateral movement therebetween, this allows a slack to the inner layer 120 for being guided by a reinforced apex into proper position with the apex 128 inserted into the cleft and the surrounding left and right-side material 126 respectively following the curvatures of the left and right side glutes as this is the path of least resistance.

Once the opening 118 is formed, due to the fact that the material 120 is cut, the longitudinal tension T1 along the opening 118 disappears, thus the lateral tension T2 is no longer the same in the material portions on the left and right of the opening 118, a new lateral tension is formed, represented by T3. The tension T3 is lesser than the tension T2. Prior to the formation of the opening, the relationship between tensions T1 and T2 was at a 90-degree angle (i.e. length vs. width).

Due to the tension ratio between T1 and T3 and the circular configuration of the opening 118, there is produced a radial tension TR in the left and right material portions 132A and 132B surrounding the opening 118.

A rectangular area 134 directly surrounding opening 118 is sealed. In an embodiment, the inner layer 120 receives a silicone (a layer, a strip, or embedded etc.) in area 134 which seals this material 120 within area 134 from waves. The silicone in area 34 allows for this area to seal against the skin of the left and right inner glutes along the cleft therebetween. This is convenient, as the material 124 and the opening 118 act as a funnel to receive feces therein during defecation.

Nevertheless, it is convenient for the area 134 to be completely flat and devoid of any of tension waves which avoids the feces from escaping via tunnels formed between the valleys of the tension waves and the inner gluteal skin on which area 134 seals to. As such, feces are lead directly within the opening 118 due to the apex 128 pushing the opening upwardly close to the anus and the material 134 sealing the area of the intergluteal cleft surrounding the anus as provided hereinabove. Thus, there is no spillage unto the top face of the inner layer 120.

The lateral portions 136A and 136B of the diaper 110 are sections where the top and bottom layers are glued together. In order to avoid the above discussed radial waves TR from being formed, wider lateral portions 138A and 138B of the top and bottom layer are glued together along a length of the portions 136A and 136B. As such, there is less loose material in areas 132A and 132B between the opening 118 and the portions 138A and 138B. Indeed, the foregoing flattens out the tension waves TR as it pulls back the slack in material 132A and 132B between the opening 118 and the portions 138A and 138B.

Moreover, the lateral pulling provided by new T3 when considering the additional tension provided by sections 138A and 138B as explained and in conjunction with T1 provides for a convenient opening tension on the opening 18.

It should be noted that the tension provided by sections 138A and 138B may cause the material portions 132A and 132B to curve, since the material is being pulled laterally but also downwardly. In essence, we are anchoring more material via sections 130A and 130B. This lateral and downward pull causes the material portions 132A and 132B to pull the area 134 away from the skin, and hence, a correction force is required to pull the area 134 upwardly and make sure it seals to the skin. The foregoing “upward pull” is provided by the longitudinal soft elastics 140A and 140B which run along the length L of the diaper 110 and are positioned at the lateral edges of area 134. Additional upward pull is also provided by opposite pairs of soft elastics 142A and 142B running along the width of portions 132A and 132B respectively between sections 138A, 138B and elastics 140A, 140B, respectively. Indeed, as shown in FIG. 12 the foregoing provides an angular upward pull tension T4 in areas 126 which correspond to the area 134.

The opening 118 can be closed by modifications of the c-shape of the diaper 110 depending on the size of the wearer. In certain cases, the c-shape will be more closed while other times it will be more open. In order to neutralize this variable diagonal low resistance elastics 144 are positioned between the longitudinal ends of the of the opening and the lateral ends 114A and 114B thus making sure the opening 118 remains open for receipt of faecal matter at all times as provided herein. As such, elastics 144 compensate for the c-shape cup movement of the diaper 110.

Turning to the example of the diaper 110 shown in FIG. 13, the portions 146A and 146B of the inner layer 120 are shown to be cut in a zigzag and thus glued along this zigzag configuration on the bottom layer. Thus, the tension T2 in the area 148 of the diaper 110 will be formed by a series of vectors that are diagonal rather than being in a straight line from end to end, as the zigzag forms oppositely directed diagonal lines 150′ and 150″, each providing a similarly directed vector across the area 148. The pitch of this zigzag can be modulated in order to correspondingly modulate the diagonal tension vectors across area 148. The tension across area 148 will thus be diluted. Indeed, with this zigzag, a much greater but smaller number of waves are created thereby smoothing out the material in area 148 rather than having lesser and larger waves which are more difficult to straighten out. Thus, in this case there are so many waves that they become negligible.

Turning to FIG. 14, the contour 152 of the inner layer 120 of the diaper 110 is configured to decrease high tension lines by providing smaller areas of tension. For example, the rear end 112A can be slightly tapered 154 and the junctions 156 between the end 112A and lateral sides 114A and 114B, respectively can be rounded rather than being a corner, thus softening the tension points. Thus, smaller tension lines provide for smaller waves and these smaller waves can easily be absorbed and become negligible across the top surface 158. The junctions 156 are cut in a zigzag shape to provide the benefits described for FIG. 13. As explained for FIG. 11, the lateral glued end sections 160 at the level of the opening 118 are larger in order to anchor more material for the benefits previously described. In this example, the sections 160 are also in zigzag configurations that are larger (more inwardly glued, thus providing larger triangular indentations) than junctions 56 as well as the front end sections 162 which do not have the opening 118 interposed therebetween but rather a contiguous uninterrupted area 164.

The opening 118 is surrounded by a silicone line 166 that provides an upward vector towards the anus at the same time the tension of sections 160 provide a lateral pull on the opening thereby causing the silicone line 166 to move upwards and outwards towards edges 114A and 114B thus sealingly gripping the inner gluteal skin circumscribing the anus thus feces flows into the pocket and not between the inner layer 120 and the skin.

In the event that a wearer defecates the feces will seal the inner layer or liner 120 with the absorption core making it very likely that if the user subsequently urinates the urine will not be able to reach the core and will spill. The diaper 110 of FIG. 15 solves this issue. An air laid paper 168 is provided at the front end portion of the diaper 110 to quicky absorb the urine and lead it to the absorbent core. The air laid paper 168 is positioned between the liner 120 and the core.

The diapers can be made of a variety of suitable materials and designed in a variety of suitable configurations within the context of the present disclosure.

The various features described herein can be combined in a variety of ways within the context of the present disclosure so as to provide still other embodiments. As such, the embodiments are not mutually exclusive. Moreover, the embodiments discussed herein need not include all of the features and elements illustrated and/or described and thus partial combinations of features can also be contemplated. Furthermore, embodiments with less features than those described can also be contemplated. It is to be understood that the present disclosure is not limited in its application to the details of construction and parts illustrated in the accompanying drawings and described hereinabove. The disclosure is capable of other embodiments and of being practiced in various ways. It is also to be understood that the phraseology or terminology used herein is for the purpose of description and not limitation. Hence, although the present disclosure has been provided hereinabove by way of non-restrictive illustrative embodiments thereof, it can be modified, without departing from the scope, spirit and nature thereof and of the appended claims.

What is claimed is:

1. A diaper comprising:
an outer side and an opposite inner side comprising an absorbent part; and
a top layer defining a peripheral border thereof and being sealed to the inner side about the peripheral border thereof and defining a top layer area delimited by the peripheral border overlying at least a portion of the absorbent part and being unconnected thereto, the top layer area, the absorbent part and the peripheral border defining a pouch therebetween, the pouch defining a pouch cavity therein, the top layer area defining a body contacting surface and providing an opening for access to the pouch cavity;
wherein when the diaper is worn by a user, the opening is provided to be positioned about the anus of the user for providing for faecal matter to enter the pouch cavity during defecation.

2. A diaper according to claim 1, wherein the opening is provided along a central line area of the top layer area.

3. A diaper according to any one of claim 1 or 2, wherein the top layer area comprises adhesive at each lateral side of the opening, the adhesive providing for adhering the user's skin at an inner side of each buttock within the intergluteal cleft thereby bringing the opening in close proximity to the anus to receive faecal matter directly therein.

4. A diaper according to claim 3, wherein the adhesive is in the form of a pair of longitudinal strips along a longitudinal length of the top layer area so as to be removably sealed along a longitudinal length of the user's skin.

5. A diaper according to any one of claim 3 or 4, wherein the adhesive provides a seal with the user's skin about each lateral side of the anus and the opening substantially blocking faecal matter from flowing onto an outer surface of the top layer area beyond the seal thereby allowing faecal matter to flow into the opening.

6. A diaper according to claim 5, wherein the adhesive for adhering the user's skin at an inner side of each buttock within the intergluteal cleft is covered by a removable liner prior to use.

7. A diaper according to any of claims 1 to 6, comprising adhesive on the top layer for providing the top layer for adhering to the user's skin directly on each buttock outside the intergluteal cleft so as to maintain the diaper in position during movement of the user.

8. A diaper according to claim 7, wherein the adhesive for adhering to the user's skin directly on each buttock outside the intergluteal cleft is provided in the form of elongate strips on the top layer for providing substantial areas of the top layer without any adhesive.

9. A diaper according to any one of claim 7 or 8, wherein the adhesive for adhering to the user's skin directly on each buttock outside the intergluteal cleft is covered by a removable liner prior to use.

10. A diaper according to any one of claims 1, wherein the top layer area comprises a tear line for being selectively torn along a length thereof to selectively form the opening.

11. A diaper according to claim 10, wherein the tear line is formed along a central line area of the top layer area for being selectively torn along a desired position of the central line area and along a desired position of the central line area.

12. A diaper according to any one of claim 10 or 11, wherein the tear line comprises weak lines or perforations, the tear line defining a central portion interposed by front and rear portions, wherein the central portion has a greater number of the weak lines or the perforations than either one of the front and rear portions.

13. A diaper according to any one of claims 1 to 12, wherein the top layer provides for allowing a front part of the absorbent part to be uncovered and thus in direct contact with the user for receiving urine discharge to be absorbed, thereby substantially avoiding urine discharge from into the pouch and mixing with the feces collected therein.

14. A diaper according to claim 13, wherein the pouch defines a front part and a rear part, the front part defining a pair of front separate fingers defining an inwardly recessed portion therebetween exposing the front portion of the absorbent part in direct contact with the user for receiving urine discharge to be absorbed.

15. A diaper according to claim 1, wherein the top layer comprises adhesive for adhering to the users skin and for adhesively sealing the inner side when folding the diaper after use thereby sealing the pouch containing the collected faecal matter.

16. A diaper according to any one of claims 1 to 15, wherein the inner side defines a length and width thereof, wherein the inner side comprises a longitudinal tension along the length thereof that is greater than the a lateral tension along the width thereof.

17. A diaper according to any one of claims 1 to 16, wherein the top layer area defines a length thereof and forms waves defining longitudinal peaks and valleys along the length thereof, wherein the opening is provided at a selected one of the longitudinal peaks.

18. A diaper according to any one of claims 1 to 17, wherein the peripheral boundary of the top layer defines lateral sides thereof, wherein at least a portion of the lateral sides of the peripheral boundary are cut in a zigzag configuration.

19. A diaper according to any one of claims 1 to 18, wherein the top layer comprises soft elastic elements running along a length thereof.

20. A diaper according to any one of claims 1 to 19, wherein the top layer area comprises silicone circumscribing the opening.
